(19) United States
(12) Patent Application Publication
(10) Pub. No.: US 2024/0284774 A1
(43) Pub. Date: Aug. 22, 2024

(54) POLYMER

(71) Applicant: SUMITOMO CHEMICAL CO., LTD., Tokyo (JP)

(72) Inventor: Michal Maciejczyk, Godmanchester (GB)

(73) Assignee: SUMITOMO CHEMICAL CO., LTD., Tokyo (JP)

(21) Appl. No.: 18/291,545

(22) PCT Filed: Jul. 25, 2022
(86) PCT No.: PCT/EP2022/070841

§ 371 Date: Jan. 23, 2024

(30) Foreign Application Priority Data
Jul. 27, 2021 (GB) 2110798.2

(51) Int. Cl. H10K 85/10; H10K 30/30; H10K 30/85; H10K 39/00

(52) U.S. Cl. CPC H10K 85/151; H10K 30/30; H10K 30/85; H10K 85/113; H10K 39/601

(57) ABSTRACT

A polymer comprising a donor repeat unit and an acceptor repeat unit wherein the acceptor repeat unit comprise a repeat unit of formula (I): A¹ is selected from formula (IIa): formula (lib); O; S; and NR¹ wherein R¹ is H or a substituent: (Ha) (lib) Ar³ is a monocyclic or polycyclic aromatic group; X¹ and X² are each independently selected from N and CR² wherein R² in each occurrence is H or a substituent with the proviso that at least one of X¹ and X² is selected from N and CR² wherein R² is an electron withdrawing group; Ar¹ is selected from pyrrole, benzene, pyridine and 1,4-diazine; A² is O, S, SO₂, NR¹, PR¹, C(R³)² and Si(R³)2 wherein R³ in each occurrence is independently H or a substituent; and Ar² is a monocyclic or polycyclic aromatic group.

### BACKGROUND

Embodiments of the present disclosure relate to polymers and more specifically, but not by way of limitation, to polymer containing an electron-accepting unit and an electron-donating unit, the polymers being suitable for use as an electron-donating material or an electron-accepting material in a photoresponsive device.

Ding et al, “Indenone-fused N-heteroacenes” J. Mater. Chem. C, 2019, 7, 14314-14319 discloses ambipolar organic semiconductors based on indenone-fused azaacenes.

JP2014181189 discloses indoloquinoxaline compounds.

### SUMMARY

In some embodiments, the present disclosure provides a polymer comprising a donor repeat unit and an acceptor repeat unit wherein the acceptor repeat unit comprise a repeat unit of formula (I):

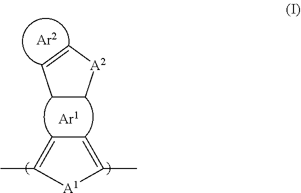

A¹ is selected from formula (IIa): formula (IIb); O; S; and NR¹ wherein R¹ is H or a substituent:

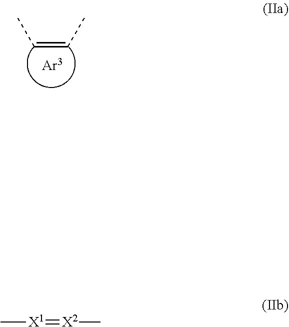

Ar³ is a monocyclic or polycyclic aromatic group which is unsubstituted or substituted with one or more substituents.

X¹ and X² are each independently selected from N and CR² wherein R² in each occurrence is H or a substituent with the proviso that at least one of X¹ and X² is selected from N and CR² wherein R² is an electron withdrawing group.

Ar¹ is selected from pyrrole, benzene, pyridine and 1,4-diazine, each of which is unsubstituted or substituted with one or more substituents.

A² is O, S, SO₂, NR¹, PR¹, C(R³)₂ and Si(R³)₂ wherein R³ in each occurrence is independently H or a substituent.

Ar² is a monocyclic or polycyclic aromatic group which is unsubstituted or substituted with one or more substituents.

Optionally, A¹ is a group of formula (IIa) and Ar³ is a monocyclic heteroaromatic group which is unsubstituted or substituted with one or more substituents.

Optionally, Ar³ is thiadiazole.

Optionally, Ar³ is 1,4-diazine which is unsubstituted or substituted with one or more substituents.

Optionally, A¹ is a group of formula (IIa) and Ar³ is a polycyclic heteroaromatic group which is unsubstituted or substituted with one or more substituents.

Optionally, Ar³ is a group of formula (III):

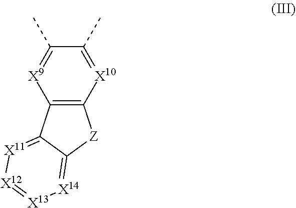

X⁹ and X¹⁰, are each independently selected from N and CR³.

X¹¹, X¹², X¹³ and X¹⁴ are each independently selected from N and CR³ with the proviso that at least one of X¹¹, X¹², X¹³ and X¹⁴ is CR³.

Z is selected from O, S, SO₂, NR¹, PR¹, C(R⁴)₂, Si(R⁴)₂ C═O, C═S and C═C(R⁵)₂ wherein R⁴ in each occurrence is independently H or a substituent and R⁵ in each occurrence is an electron-withdrawing group.

Optionally, the repeat unit of formula (I) has formula (Ia):

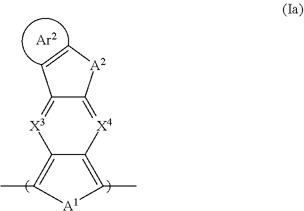

wherein X³ and X⁴ are each independently selected from N and CR³ wherein R³ in each occurrence is independently H or a substituent.

Optionally, X³ and X⁴ are each N.

Optionally, Ar² is a six-membered aromatic or heteroaromatic group wherein each ring atom is selected from C and N and wherein Ar² is unsubstituted or substituted with one or more substituents.

Optionally, the repeat unit of formula (I) has formula (Ib):

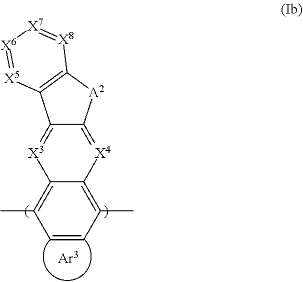

wherein X³, X⁴, X⁵, X⁶, X⁷, X⁸, X⁹ and X¹⁰ are each independently selected from N and CR³ wherein R³ in each occurrence is independently H or a substituent.
Optionally, X³ and X⁴ are N.

Optionally, the donor repeat units are selected from one or more units of formulae (IVa)-(IVt) as described below.

Optionally, the R⁵⁰ groups are linked of formula (IVa) are linked.

Optionally, the polymer comprises a repeat unit of formula (IVa-1):

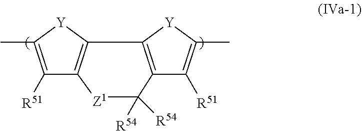

Optionally, the polymer has an absorption peak of greater than 1000 nm.

According to some embodiments, the present disclosure provides a composition comprising an electron donor and an electron acceptor wherein the electron donor is a polymer as described herein.

According to some embodiments, the present disclosure provides an organic electronic device comprising an active layer comprising a polymer or composition as described herein.

Optionally, the organic electronic device is an organic photoresponsive device comprising a bulk heterojunction layer disposed between an anode and a cathode and wherein the bulk heterojunction layer comprises a composition as described herein.

Optionally, the organic photoresponsive device is an organic photodetector.

According to some embodiments, the present disclosure provides a photosensor comprising a light source and an organic photodetector as described herein, wherein the photosensor is configured to detect light emitted from the light source.

Optionally, the light source emits light having a peak wavelength of >1000 nm.

Optionally, the light source emits light having a peak wavelength of >1200 nm.

According to some embodiments, the present disclosure provides a formulation comprising a polymer or composition as described herein dissolved or dispersed in one or more solvents.

According to some embodiments, the present disclosure provides a method of forming an organic electronic device as described herein wherein formation of the active layer comprises deposition of a formulation as described herein onto a surface and evaporation of the one or more solvents

### DESCRIPTION OF DRAWINGS

The disclosed technology and accompanying figures describe some implementations of the disclosed technology.

FIG. 1 illustrates an organic photoresponsive device according to some embodiments; and

FIG. 2 shows absorption spectra in toluene solution for a polymer according to an embodiment of the present disclosure and comparative polymers.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to a layer “over” another layer when used in this application means that the layers may be in direct contact or one or more intervening layers are may be present. References to a layer “on” another layer when used in this application means that the layers are in direct contact. References to a specific atom include any isotope of that atom unless specifically stated otherwise.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

A polymer as described herein may be provided in a bulk heterojunction layer of a photoresponsive device, preferably a photodetector, in which the bulk heterojunction layer is disposed between an anode and a cathode.

The bulk heterojunction layer comprises an electron donor material and an electron acceptor material wherein at least one of the electron donor material and the electron acceptor material is a polymer having an electron-accepting repeat unit of formula (I) and an electron-donating repeat unit:

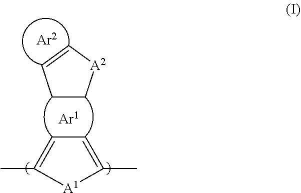

A¹ is selected from formula (IIa): formula (IIb); O; S; and NR¹ wherein R¹ is H or a substituent:

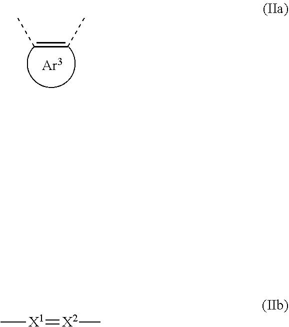

R¹ is preferably H; C₁₋₁₂ alkyl wherein a C atom of the alkyl other than the C atom bound to N may be replaced with O, S, CO or COO and one or more H atoms may be replaced with F; and an aromatic or heteroaromatic group, preferably phenyl, which may be unsubstituted or substituted with one or more substituents. Exemplary substituents of the aromatic or heteroaromatic group are F, CN, NO₂, and C₁₋₁₂ alkyl wherein one or more non-adjacent C atoms may be replaced with O, S, NR⁷ wherein R⁷ is a C₁₋₁₂ hydrocarbyl group, COO or CO and one or more H atoms of the alkyl may be replaced with F. R⁷ may be, for example, a C₁₋₁₂ alkyl; unsubstituted phenyl; or phenyl substituted with one or more C₁₋₆ alkyl groups.

If a C atom of an alkyl group as described anywhere herein is replaced with another atom or group, the replaced C atom may be a terminal C atom of the alkyl group or a non-terminal C-atom.

By “non-terminal C atom” of an alkyl group as used anywhere herein means a C atom other than the C atom of the methyl group at the end of an n-alkyl chain or the C atoms of the methyl groups at the ends of a branched alkyl chain.

If a terminal C atom of a group as described anywhere herein is replaced then the resulting group may be an anionic group comprising a countercation, e.g. an ammonium or metal countercation, preferably an ammonium or alkali metal cation.

A C atom of an alkyl group which is replaced with another atom or group as described anywhere herein is preferably a non-terminal C atom.

Preferably, A¹ is a group of formula (IIa).

In some embodiments, A¹ is a group of formula (IIa) and Ar³ is a monocyclic heteroaromatic group which is unsubstituted or substituted with one or more substituents. Optionally according to these embodiments, Ar³ is thiadiazole or 1,4-diazine which is unsubstituted or substituted with one or more substituents.

In some embodiments, A¹ is a group of formula (IIa) and Ar³ is a polycyclic heteroaromatic group which is unsubstituted or substituted with one or more substituents. Optionally according to these embodiments, Ar³ is a group of formula (III):

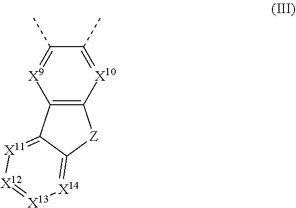

X⁹ and X¹⁰, are each independently selected from N and CR³ wherein R³ is H or a substituent.

X¹¹, X¹², X¹³ and X¹⁴ are each independently selected from N and CR³ with the proviso that at least one of X¹¹, X¹², X¹³ and X¹⁴ is CR³.

Z is selected from O, S, SO₂, NR¹, PR¹, C(R⁴)₂, Si(R⁴)₂ C═O, C═S and C═C(R⁵)₂ wherein R¹ is as described above; R⁴ in each occurrence is independently H or a substituent; and R⁵ in each occurrence is an electron-withdrawing group.

Where present, preferred substituents of Ar³, for example R³, are selected from F; CN; NO₂; C₁₋₂₀ alkyl wherein one or more non-adjacent C atoms may be replaced with O, S, NR⁷, COO or CO and one or more H atoms of the alkyl may be replaced with F; and phenyl which is unsubstituted or substituted with one or more substituents, optionally one or more C₁₋₁₂ alkyl groups wherein one or more non-adjacent C atoms may be replaced with O, S, NR⁷, COO or CO and one or more H atoms of the alkyl may be replaced with F.

Preferably, each R⁴ is independently selected from C₁₋₂₀ alkyl wherein one or more non-adjacent C atoms may be replaced with O, S, NR⁷, COO or CO and one or more H atoms of the alkyl may be replaced with F; and an aromatic or heteroaromatic group, preferably phenyl, which may be unsubstituted or substituted with one or more substituents. Exemplary substituents of the aromatic or heteroaromatic group are F, CN, NO₂, and C₁₋₁₂ alkyl wherein one or more non-adjacent C atoms may be replaced with O, S, NR⁷, COO or CO and one or more H atoms of the alkyl may be replaced with F.

Preferably, each R⁵ is CN.

With reference to formula (IIb), X¹ and X² are each independently selected from N and CR² wherein R² in each occurrence is H or a substituent with the proviso that at least one of X¹ and X² is selected from N and CR² wherein R² is an electron withdrawing group.

In some embodiments, the group of formula (IIb) has formula —CR²═CR²— wherein at least one R², preferably each R², is an electron-withdrawing group.

In some embodiments, the group of formula (IIb) has formula —CR²═N— wherein R² is H or a substituent.

Optionally, each R² is independently selected from H; F; CN; NO₂; C₁₋₂₀ alkyl wherein one or more non-adjacent C atoms may be replaced with O, S, NR⁷, COO or CO and one or more H atoms of the alkyl may be replaced with F; and phenyl which is unsubstituted or substituted with one or more substituents, optionally one or more C₁₋₁₂ alkyl groups wherein one or more non-adjacent C atoms may be replaced with O, S, NR⁷, COO or CO and one or more H atoms of the alkyl may be replaced with F.

Preferred electron-withdrawing groups R² are F, CN, NO₂ and C₁₋₁₂ alkyl wherein one or more H atoms are replaced with F.

Ar¹ is selected from pyrrole; 1,4-diazine; pyridine which is unsubstituted or substituent with a substituent; or benzene which is unsubstituted or substituted with one or two substituents.

Where present, substituents of pyridine or benzene groups Ar¹ are preferably selected from substituents R³ described above.

A² is O, S, SO₂, NR¹, PR¹, C(R⁴)₂ and Si(R⁴)₂ wherein R¹ and R⁴ are as described above.

Preferably, A² is O, S or NR¹.

Ar² is a monocyclic or polycyclic aromatic group which is unsubstituted or substituted with one or more substituents. Preferably, Ar² is a six-membered aromatic or heteroaromatic group wherein each ring atom is selected from C and N and wherein Ar² is unsubstituted or substituted with one or more substituents. Preferred substituents are as described with respect to R³.

In some preferred embodiments, the repeat unit of formula (I) has formula (Ia):

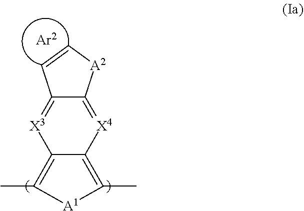

wherein X³ and X⁴ are each independently selected from N and CR³ wherein R³ is as described above.

In some preferred embodiments, the repeat unit of formula (I) has formula (Ib):

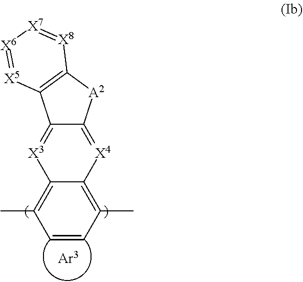

wherein X⁵, X⁶, X⁷ and X⁸ are each independently selected from N and CR³ wherein R³ in each occurrence is independently H or a substituent.

Preferably, each of X³ and X⁴ of formulae (la) and (Tb) are N.

Exemplary repeat units of formula (I) are illustrated below, wherein each unsubstituted aromatic carbon atom may optionally be substituted with a substituent R³ as described above:

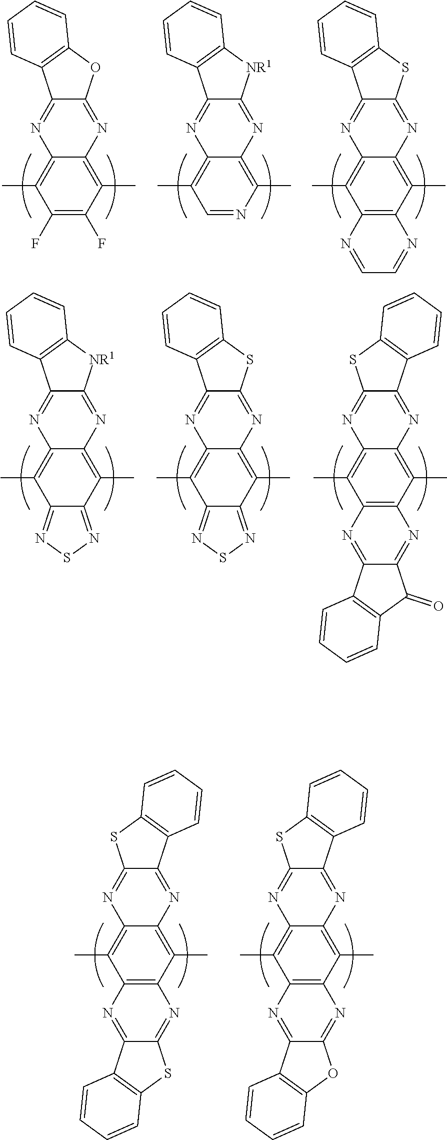

In some embodiments, the polymer has an absorption peak greater than 1000 nm, optionally greater than 1200 nm.

Unless stated otherwise, absorption spectra of materials as described herein are measured using a Cary 5000 UV-VIS-NIR Spectrometer. Measurements were taken from 175 nm to 3300 nm using a PbSmart NIR detector for extended photometric range with variable slit widths (down to 0.01 nm) for optimum control over data resolution.

Absorption data are obtained by measuring the intensity of transmitted radiation through a solution sample. Absorption intensity is plotted vs. incident wavelength to generate an absorption spectrum. A method for measuring film absorption, may comprise measuring a 15 mg/ml solution in a quartz cuvette and comparing to a cuvette containing the solvent only.

Unless stated otherwise, absorption data as provided herein is as measured in toluene solution.

The polymer contains an electron acceptor unit of formula (I) and an electron donor repeat unit.

The polymer may contain only one electron donor repeat unit. The polymer may contain two or more different electron donor repeat units.

Preferably, each repeat unit of formula (I) is directly bound to a donor repeat unit. More preferably, the polymer comprises alternating donor and acceptor repeat units.

The repeat unit of formula (I) may be the only electron acceptor repeat unit of the polymer. The polymer may contain one or more further electron acceptor repeat units.

The, or each, electron donor (p-type) repeat unit has a HOMO deeper (further from vacuum) than a LUMO of the electron acceptor (n-type) repeat unit of formula (I) and, if present, any further electron acceptor repeat units.

The electron-accepting repeat unit of formula (I) has a LUMO level that is deeper (i.e. further from vacuum) than the LUMO of the, or each, electron-donating repeat unit, preferably at least 1 eV deeper. The LUMO levels of repeat units of formula (I) and electron-donating repeat units may be as determined by modelling the LUMO level of each repeat unit, in which bonds to adjacent repeat units are replaced with bonds to a hydrogen atom. Modelling may be performed using Gaussian09 software available from Gaussian using Gaussian09 with B3LYP (functional) and LACVP* (Basis set).

Optionally, the gap between the HOMO level of the p-type donor repeat unit and the LUMO level of the n-type acceptor repeat unit of formula (I) is less than 1.4 eV. Unless stated otherwise, HOMO and LUMO levels of materials as described herein are as measured by square wave voltammetry (SWV).

In SWV, the current at a working electrode is measured while the potential between the working electrode and a reference electrode is swept linearly in time. The difference current between a forward and reverse pulse is plotted as a function of potential to yield a voltammogram. Measurement may be with a CHI 660D Potentiostat.

The apparatus to measure HOMO or LUMO energy levels by SWV may comprise a cell containing 0.1 M tertiary butyl ammonium hexafluorophosphate in acetonitrile; a 3 mm diameter glassy carbon working electrode; a platinum counter electrode and a leak free Ag/AgCl reference electrode.

Ferrocene is added directly to the existing cell at the end of the experiment for calculation purposes where the potentials are determined for the oxidation and reduction of ferrocene versus Ag/AgCl using cyclic voltammetry (CV).

The sample is dissolved in toluene (3 mg/ml) and spun at 3000 rpm directly on to the glassy carbon working electrode.

LUMO=4.8-E ferrocene (peak to peak average)−E reduction of sample (peak maximum).

HOMO=4.8-E ferrocene (peak to peak average)+E oxidation of sample (peak maximum).

A typical SWV experiment runs at 15 Hz frequency; 25 mV amplitude and 0.004 V increment steps. Results are calculated from 3 freshly spun film samples for both the HOMO and LUMO data.

In some embodiments, the bulk heterojunction layer contains only one electron donor material and only one electron acceptor material, at least one of the donor and acceptor comprising a polymer as described herein.

In some embodiments, the bulk heterojunction layer contains two or more electron donor materials and/or two or more electron acceptor materials.

In some embodiments, the weight of the donor material(s) to the acceptor material(s) is from about 1:0.5 to about 1:2, preferably about 1:1.1 to about 1:2.

Preferably, the polystyrene-equivalent number-average molecular weight (Mn) measured by gel permeation chromatography of the polymer is in the range of about 5×10³ to 1×10⁸, and preferably 1×10⁴ to 5×10⁶. The polystyrene-equivalent weight-average molecular weight (Mw) of the polymer may be 1×10³ to 1×10⁸, and preferably 1×10⁴ to 1×10⁷.

### Electron-Donating Repeat Unit

Electron-donating repeat units D are preferably in each occurrence a monocyclic or polycyclic heteroaromatic group which contains at least one furan or thiophene and which may be unsubstituted or substituted with one or more substituents. Preferred electron-donating units D are monocyclic thiophene or furan or a polycyclic donor wherein each ring of the polycyclic donor includes thiophene or furan rings and, optionally, one or more of benzene, cyclopentane, or a six-membered ring containing 5 C atoms and one of N and O atoms.

Optionally, electron donating units D are selected from formulae (IVa)-(IVt), or a combination thereof:

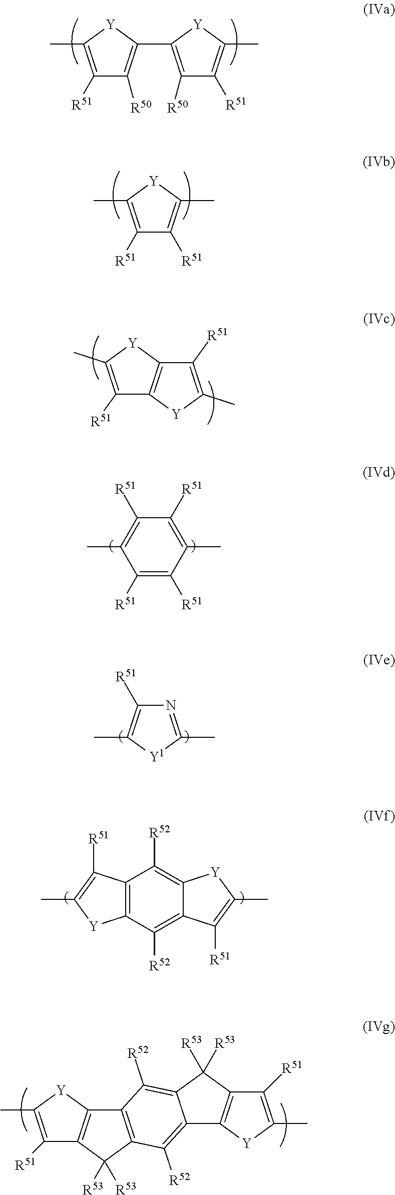

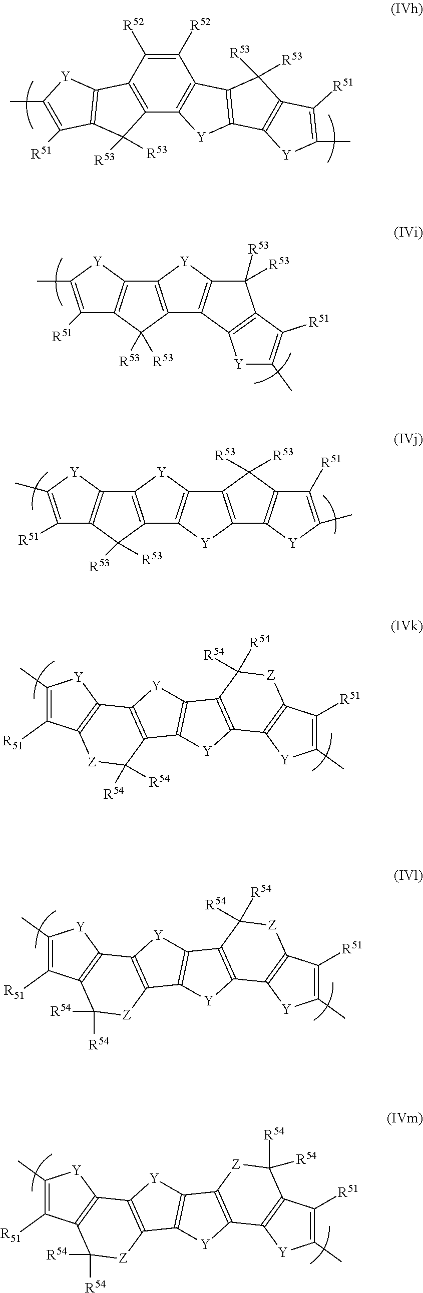

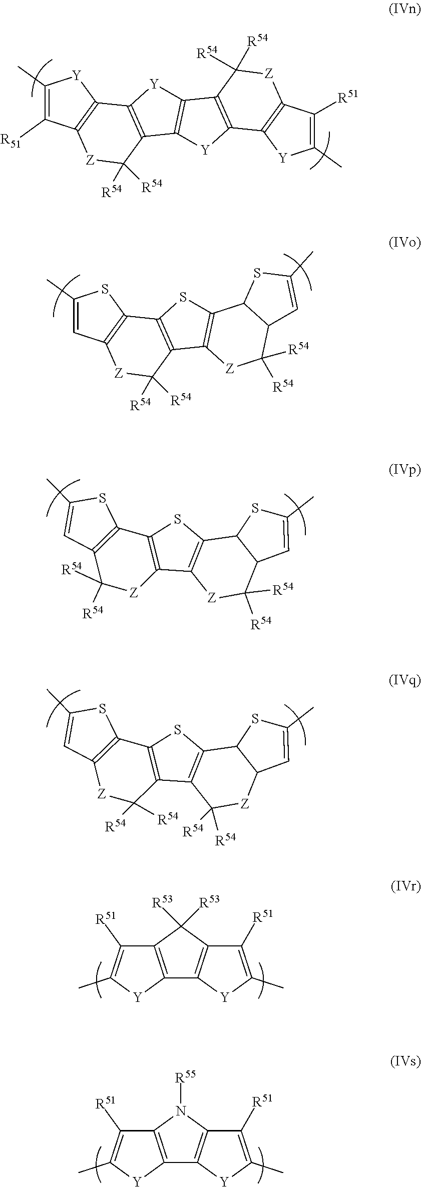

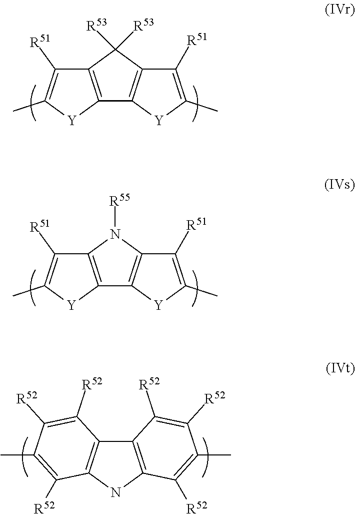

wherein Y and Y¹ in each occurrence is independently O or S, Z¹ in each occurrence is O, S, NR⁵⁵ or C(R⁵⁴)₂; R⁵⁰, R⁵¹, R⁵² R⁵⁴ and R⁵⁵ independently in each occurrence is H or a substituent wherein R⁵⁰ groups may be linked to form a ring; and R⁵³ independently in each occurrence is a substituent.

The electron-donating repeat unit may comprise a plurality of groups selected from formulae (IVa)-(IVt) linked in a continuous chain.

Preferably, the polymer comprises an electron-donating repeat unit comprising or consisting of formula (IVa-1):

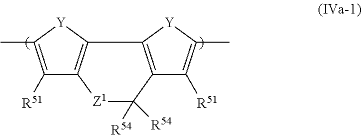

wherein Y, Z¹, R⁵¹ and R⁵⁴ are as described above. The electron-donating repeat unit may comprise or consist of a chain of units of formula (IVa-1), e.g. 2 or 3 linked units of formula (IVa-1).

Y of formula (IVa) is preferably S.

Z of formula (IVa) is preferably O or S.

In some preferred embodiments, the only electron-donating repeat unit or units of the polymer are units of formula (IVa-1).

In some preferred embodiments, the polymer comprises two or more different electron-donating units, preferably two or more different electron donating units selected from formulae (IVa)-(IVt).

The polymer may comprise a chain of directly linked electron-donating units, in which case each unit may be linked in any orientation. For example, in the case where each electron-donating unit D is a group of formula (IVa-1) and n is 2, -[D]ₙ- may be selected from any of:

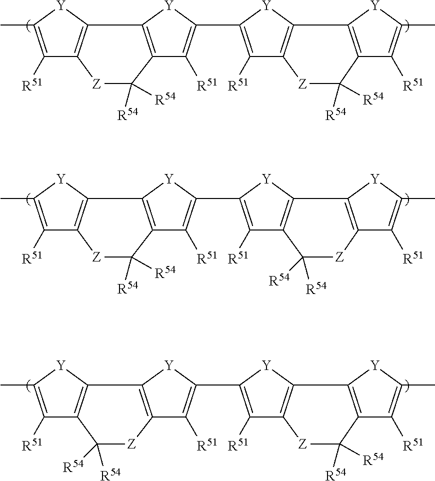

Optionally, R⁵⁰, R⁵¹ and R⁵² independently in each occurrence are selected from H; F; C₁₋₂₀ alkyl wherein one or more non-adjacent C atoms may be replaced with O, S, COO or CO and one or more H atoms of the alkyl may be replaced with F; and an aromatic or heteroaromatic group Ar³ which is unsubstituted or substituted with one or more substituents.

In some embodiments, Ar³ may be an aromatic group, e.g. phenyl.

The one or more substituents of Ar³, if present, may be selected from C₁₋₁₂ alkyl wherein one or more non-adjacent C atoms may be replaced with O, S, NR⁷, COO or CO and one or more H atoms of the alkyl may be replaced with F.

Preferably, each R⁵⁴ is selected from the group consisting of:
H;
F;
linear, branched or cyclic C₁₋₂₀ alkyl wherein one or more non-adjacent C atoms may be replaced by O, S, NR⁷, CO or COO wherein R⁷ is a C₁₋₁₂ hydrocarbyl and one or more H atoms of the C₁₋₂₀ alkyl may be replaced with F; and
a group of formula -(Ak)u-(Ar⁷)v wherein Ak is a C₁₋₁₂ alkylene chain in which one or more non-adjacent C atoms may be replaced with O, NR⁷, S, CO or COO; u is 0 or 1; Ar⁷ in each occurrence is independently an aromatic or heteroaromatic group which is unsubstituted or substituted with one or more substituents; and v is at least 1, optionally 1, 2 or 3.

Substituents of Ar⁷, if present, are preferably selected from F; Cl; NO₂; CN; and C₁₋₂₀ alkyl wherein one or more non-adjacent C atoms may be replaced with O, S, NR⁷, CO or COO.

Preferably, Ar⁷ is phenyl.

Preferably, each R⁵¹ is H.

Optionally, R⁵³ independently in each occurrence is selected from C₁₋₂₀ alkyl wherein one or more non-adjacent C atoms may be replaced with O, S, NR⁷, COO or CO and one or more H atoms of the alkyl may be replaced with F; and phenyl which is unsubstituted or substituted with one or more substituents, optionally one or more C₁₋₁₂ alkyl groups wherein one or more non-adjacent C atoms may be replaced with O, S, NR⁷, COO or CO and one or more H atoms of the alkyl may be replaced with F.

Preferably, R⁵⁵ is H or C₁₋₃₀ hydrocarbyl group

Preferably, each R⁵⁰ is a substituent.

Exemplary repeat units of formula (IIa-1) include, without limitation:

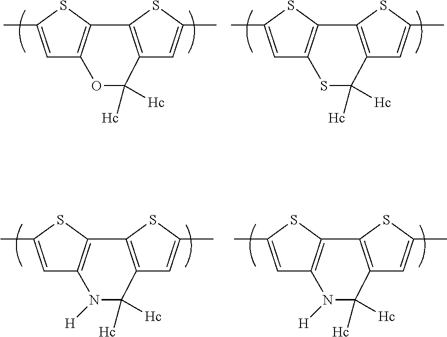

wherein He in each occurrence is independently a C₁₋₂₀ hydrocarbyl group, e.g. C₁₋₂₀ alkyl, unsubstituted aryl, or aryl substituted with one or more C₁₋₁₂ alkyl groups. The aryl group is preferably phenyl.

### Polymer Synthesis and Monomers

A polymer as described herein may be formed by polymerising a monomer for forming electron-donating repeat unit D and a monomer for forming the electron-accepting repeat unit of formula (I). The polymerisation method includes, without limitation, methods for forming a carbon-carbon bond between an aromatic carbon atom of an electron-donating unit D and an aromatic carbon atom of an electron-accepting unit (I).

In some embodiments, formation of the polymer comprises polymerisation of a monomer of formula (Xa) and a monomer of formula (Xb):

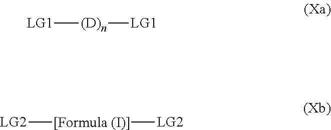

In some embodiments, formation of the polymer comprises polymerisation of a monomer of formula (Xc) and a monomer of formula (Xd):

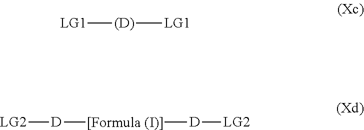

LG1 is a first leaving group bound to an aromatic carbon atom.

LG2 is a second leaving group bound to an aromatic carbon atom which is different from LG1.

A carbon-carbon bond is formed during polymerisation between aromatic carbon atoms to which LG1 and LG2 are bound.

It will be understood that a repeat unit as described anywhere herein may be formed from a monomer comprising or consisting of the repeat unit and leaving groups. For example, polymerisation of Formula (Xd) forms a repeat unit including D and Formula (I) repeat units.

Optionally, LG1 is selected from one of group (a) and group (b), and LG2 is selected from the other of group (a) and group (b):
(a) halogen or —OSO₂R⁸ wherein R⁸ is an optionally substituted C₁₋₁₂ alkyl group or optionally substituted aryl group;
(b) boronic acid and esters thereof; and —SnR⁹₃ wherein R⁹ independently in each occurrence is a C₁₋₁₂ hydrocarbyl group.

Suitable polymerisation methods include, without limitation, Suzuki polymerisation and Stille polymerisation. Suzuki polymerisation is described in, for example, WO 00/53656.

In some embodiments, each LG1 may be one of: (i) a halogen or —OSO₂R⁶; or (ii), a boronic acid or ester, and each LG2 may be the other of (i) and (ii).

In some embodiments, each LG1 may be one of: (i) a halogen or —OSO₂R⁶; and (iii) —SnR⁹₃, and each LG2 may be the other of (i) and (iii).

Optionally, R⁶ in each occurrence is independently a C₁₋₁₂ alkyl group which is unsubstituted or substituted with one or more F atoms; or phenyl which is unsubstituted or substituted with one or more F atoms.

—OSO₂R⁶ is preferably tosylate or triflate.

Exemplary boronic esters have formula (VIII):

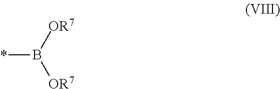

wherein R⁷ in each occurrence is independently a C₁₋₂₀ alkyl group, * represents the point of attachment of the boronic ester to an aromatic ring of the monomer, and the two groups R⁷ may be linked to form a ring which is unsubstituted or substituted with one or more substituents, e.g. one or more C₁₋₆ alkyl groups.

Optionally, R⁷ independently in each occurrence is selected from the group consisting of C₁₋₁₂ alkyl; unsubstituted phenyl; and phenyl substituted with one or more C₁₋₆ alkyl groups.

In a preferred embodiment, the two groups R⁷ are linked, e.g. to form:

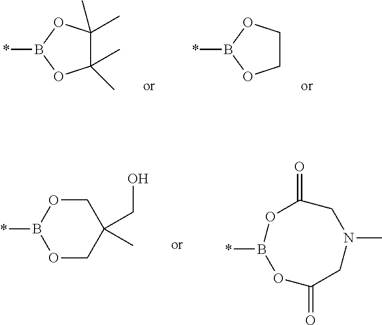

A halogen leaving group is preferably Br or I.

### Electron Accepting Material

A bulk heterojunction layer as described herein comprises an electron-donating material and an electron-accepting material wherein one of the electron-donating material and electron-accepting material is a polymer comprising a repeat unit of formula (I).

Preferably, the polymer is the electron-donating material. The electron-accepting material may be selected from any electron-accepting material known to the skilled person including, without limitation, non-fullerene acceptors and fullerene acceptors.

Preferably, the polymer is the electron-donating material and has a type II interface with the electron-accepting material, i.e. the electron-donating material has a shallower HOMO and LUMO that the corresponding HOMO and LUMO levels of the electron-accepting material. Preferably, the polymer has a LUMO that is at least 0.2 eV shallower (i.e. closer to vacuum level), optionally at least 0.3 or 0.4 eV shallower than the LUMO of the electron-accepting material. Preferably, the polymer has a HOMO that is at least 0.5 eV shallower, optionally at least 0.6 or 0.7 eV shallower, than the HOMO of the electron-accepting material.

Non-fullerene acceptors are described in, for example, Cheng et. al., “Next-generation organic photovoltaics based on non-fullerene acceptors”, Nature Photonics volume 12, pages 131-142 (2018), the contents of which are incorporated herein by reference, and which include, without limitation, PDI, ITIC, ITIC, IEICO and derivatives thereof, e.g. fluorinated derivatives thereof such as ITIC-4F and IEICO-4F.

Exemplary fullerene electron acceptor materials are C₆₀, C₇₀, C₇₆, C₇₈ and C₈₄ fullerenes or a derivative thereof, including, without limitation, PCBM-type fullerene derivatives including phenyl-C₆₁-butyric acid methyl ester (C₆₀PCBM), TCBM-type fullerene derivatives (e.g. tolyl-C₆₁-butyric acid methyl ester (C₆₀TCBM)), and ThCBM-type fullerene derivatives (e.g. thienyl-C₆₁-butyric acid methyl ester (C₆₀ThCBM).

Fullerene derivatives may have formula (V):

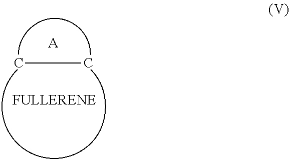

wherein A, together with the C—C group of the fullerene, forms a monocyclic or fused ring group which may be unsubstituted or substituted with one or more substituents.

Exemplary fullerene derivatives include formulae (Va), (Vb) and (Vc):

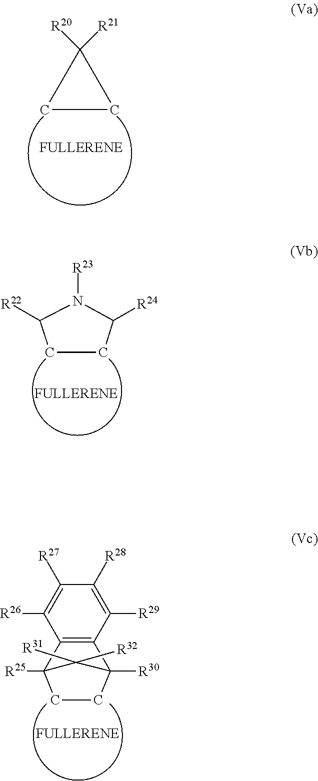

wherein R²⁰-R³² are each independently H or a substituent.

Substituents R²⁰-R³² are optionally and independently in each occurrence selected from the group consisting of aryl or heteroaryl, optionally phenyl, which may be unsubstituted or substituted with one or more substituents; and C₁₋₂₀ alkyl wherein one or more non-adjacent C atoms may be replaced with O, S, NR⁷, CO or COO and one or more H atoms may be replaced with F.

Substituents of aryl or heteroaryl, where present, are optionally selected from C₁₋₁₂ alkyl wherein one or more non-adjacent C atoms may be replaced with O, S, NR⁷, CO or COO and one or more H atoms may be replaced with F.

A composition as described herein may comprise a polymer comprising a repeat unit of formula (I) and one or more electron-accepting materials. In some embodiments, the composition comprises a non-fullerene electron accepting material and a fullerene electron-accepting material.

### Formulations

The bulk heterojunction layer may be formed by any process including, without limitation, thermal evaporation and solution deposition methods.

Preferably, the bulk heterojunction layer is formed by depositing a formulation comprising the electron donor material(s), the electron acceptor material(s) and any other components of the bulk heterojunction layer dissolved or dispersed in a solvent or a mixture of two or more solvents. The formulation may be deposited by any coating or printing method including, without limitation, spin-coating, dip-coating, roll-coating, spray coating, doctor blade coating, wire bar coating, slit coating, ink jet printing, screen printing, gravure printing and flexographic printing.

The one or more solvents of the formulation may optionally comprise or consist of benzene substituted with one or more substituents selected from chlorine, C₁₋₁₀ alkyl and C₁₋₁₀ alkoxy wherein two or more substituents may be linked to form a ring which may be unsubstituted or substituted with one or more C₁₋₆ alkyl groups, optionally toluene, xylenes, trimethylbenzenes, tetramethylbenzenes, anisole, indane and its alkyl-substituted derivatives, and tetralin and its alkyl-substituted derivatives.

The formulation may comprise a mixture of two or more solvents, preferably a mixture comprising at least one benzene substituted with one or more substituents as described above and one or more further solvents. The one or more further solvents may be selected from esters, optionally alkyl or aryl esters of alkyl or aryl carboxylic acids, optionally a C₁₋₁₀ alkyl benzoate, benzyl benzoate or dimethoxybenzene. In preferred embodiments, a mixture of trimethylbenzene and benzyl benzoate is used as the solvent. In other preferred embodiments, a mixture of trimethylbenzene and dimethoxybenzene is used as the solvent.

The formulation may comprise further components in addition to the electron acceptor, the electron donor and the one or more solvents. As examples of such components, adhesive agents, defoaming agents, deaerators, viscosity enhancers, diluents, auxiliaries, flow improvers colourants, dyes or pigments, sensitizers, stabilizers, nanoparticles, surface-active compounds, lubricating agents, wetting agents, dispersing agents and inhibitors may be mentioned.

Organic Electronic Device A polymer or composition as described herein may be provided as an active layer of an organic electronic device. In a preferred embodiment, a bulk heterojunction layer of an organic photoresponsive device, more preferably an organic photodetector, comprises a composition as described herein.

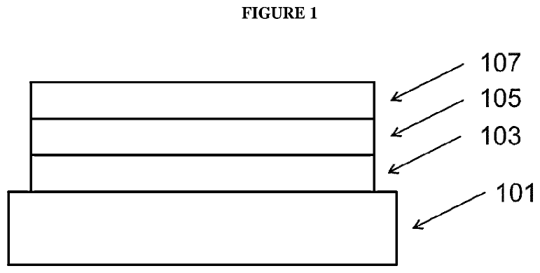

FIG. 1 illustrates an organic photoresponsive device according to some embodiments of the present disclosure. The organic photoresponsive device comprises a cathode 103, an anode 107 and a bulk heterojunction layer 105 disposed between the anode and the cathode. The organic photoresponsive device may be supported on a substrate 101, optionally a glass or plastic substrate.

Each of the anode and cathode may independently be a single conductive layer or may comprise a plurality of layers.

At least one of the anode and cathode is transparent so that light incident on the device may reach the bulk heterojunction layer. In some embodiments, both of the anode and cathode are transparent. The transmittance of a transparent electrode may be selected according to an emission wavelength of a light source for use with the organic photodetector.

FIG. 1 illustrates an arrangement in which the cathode is disposed between the substrate and the anode. In other embodiments, the anode may be disposed between the cathode and the substrate.

The organic photoresponsive device may comprise layers other than the anode, cathode and bulk heterojunction layer shown in FIG. 1. In some embodiments, a hole-transporting layer is disposed between the anode and the bulk heterojunction layer. In some embodiments, an electron-transporting layer is disposed between the cathode and the bulk heterojunction layer.

In some embodiments, a work function modification layer is disposed between the bulk heterojunction layer and the anode, and/or between the bulk heterojunction layer and the cathode.

The area of the OPD may be less than about 3 cm², less than about 2 cm², less than about 1 cm 2, less than about 0.75 cm², less than about 0.5 cm² or less than about 0.25 cm². Optionally, each OPD may be part of an OPD array wherein each OPD is a pixel of the array having an area as described herein, optionally an area of less than 1 mm², optionally in the range of 0.5 micron²-900 micron².

The substrate may be, without limitation, a glass or plastic substrate. The substrate can be an inorganic semiconductor. In some embodiments, the substrate may be silicon. For example, the substrate can be a wafer of silicon. The substrate is transparent if, in use, incident light is to be transmitted through the substrate and the electrode supported by the substrate.

The bulk heterojunction layer contains a polymer as described herein and an electron acceptor material. The bulk heterojunction layer may consist of these materials or may comprise one or more further materials, for example one or more further electron donor materials and/or one or more further electron acceptor materials.

### Applications

A circuit may comprise the OPD connected to a voltage source for applying a reverse bias to the device and/or a device configured to measure photocurrent. The voltage applied to the photodetector may be variable. In some embodiments, the photodetector may be continuously biased when in use.

In some embodiments, a photodetector system comprises a plurality of photodetectors as described herein, such as an image sensor of a camera.

In some embodiments, a sensor may comprise an OPD as described herein and a light source wherein the OPD is configured to receive light emitted from the light source. In some embodiments, the light source has a peak wavelength of at least 1000 nm, optionally in the range of 1000-1500 nm.

The present inventors have found that a material comprising an electron-accepting unit of formula (I) may be used for the detection of light at longer wavelengths, particularly 1300-1400 nm.

In some embodiments, the light from the light source may or may not be changed before reaching the OPD. For example, the light may be reflected, filtered, down-converted or up-converted before it reaches the OPD.

The organic photoresponsive device as described herein may be an organic photovoltaic device or an organic photodetector. An organic photodetector as described herein may be used in a wide range of applications including, without limitation, detecting the presence and/or brightness of ambient light and in a sensor comprising the organic photodetector and a light source. The photodetector may be configured such that light emitted from the light source is incident on the photodetector and changes in wavelength and/or brightness of the light may be detected, e.g. due to absorption by, reflection by and/or emission of light from an object, e.g. a target material in a sample disposed in a light path between the light source and the organic photodetector. The sample may be a non-biological sample, e.g. a water sample, or a biological sample taken from a human or animal subject. The sensor may be, without limitation, a gas sensor, a biosensor, an X-ray imaging device, an image sensor such as a camera image sensor, a motion sensor (for example for use in security applications) a proximity sensor or a fingerprint sensor. A 1D or 2D photosensor array may comprise a plurality of photodetectors as described herein in an image sensor. The photodetector may be configured to detect light emitted from a target analyte which emits light upon irradiation by the light source or which is bound to a luminescent tag which emits light upon irradiation by the light source. The photodetector may be configured to detect a wavelength of light emitted by the target analyte or a luminescent tag bound thereto.

### EXAMPLES

### Monomer Example 1

A monomer was prepared according to the following scheme:

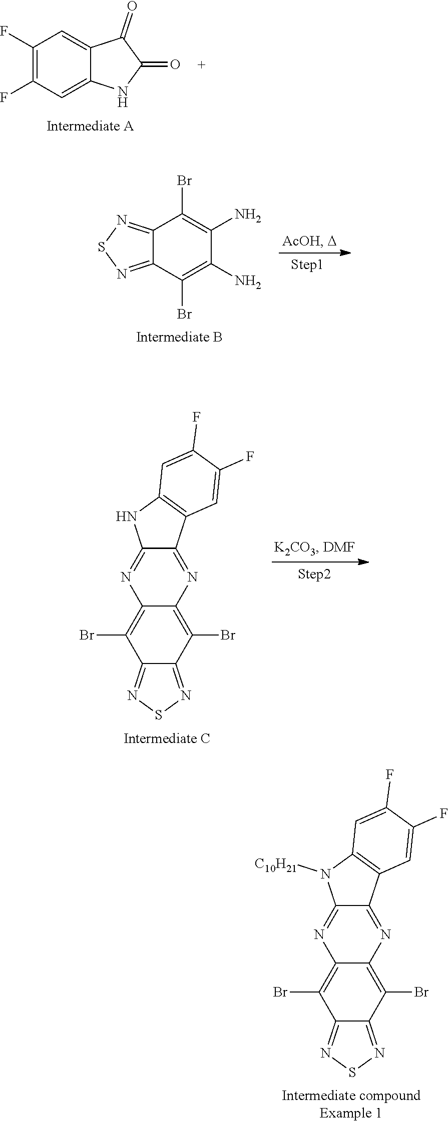

### Step 1

A mixture of glacial acetic acid (300 ml), Intermediate A (5.69 g, 31.1 mmol) and Intermediate B (9.19 g, 28.3 mmol) was, stirred at 80° C. overnight under a nitrogen atmosphere. After this time, the temperature was increased to 120° C. and stirring continued for 24 hrs. The reaction mixture was then cooled to 0° C., filtered, washed with methanol and dried overnight under vacuum to give Intermediate C (8 g), having 50% purity by HPLC. This was used in the next step without further purification.

### Step 2

A solution of Intermediate C (5.9 g, 12.5 mmol), 1-iododecane (5.33 ml, 25.0 mmol) and potassium carbonate (3.45 g, 25.0 mmol) DMF (120 ml) was heated at 60° C. for 1 hour under nitrogen. The reaction mixture was cooled to room temperature, diluted with water/ice and extracted with heptane, toluene, diethyl ether and ethyl acetate. The combined organic phases were washed with water, dried over anhydrous magnesium sulfate, filtered, evaporated and the crude product was purified by column chromatography (THF:heptane eluant). The pure fractions were filtered from warm heptane (50° C.) to give Intermediate compound 1 (0.89, 23% yield) as a purple solid.

¹H NMR (CDCl₃) 8.31 (t, 1H), 7.22 (dd, 1H), 4.44 (t, 2H), 1.98 (m, 2H), 1.42 (m, 4H), 1.23 (m, 10H), 0.83 (t, 3H)

### Polymer Example 1

Polymer Example 1, was formed by Suzuki-Miyaura polymerisation of Intermediate Compound Example 1 with two boronic ester-substituted monomers for forming electron-donating repeat units as disclosed in U.S. Pat. No. 9,512,149, the contents of which are incorporated herein by reference.

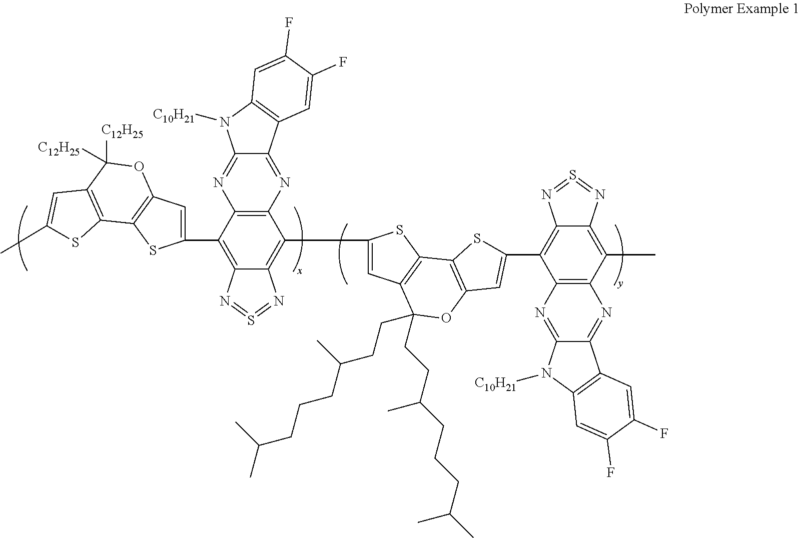

Of the two boronic ester-substituted monomers for forming electron-donating repeat units as illustrated above, one contained C₁₂H₂₅ substituents and the other contained 3,7-dimethyloctyl substituents.

The x:y molar ratio is 1:1.

### Absorption

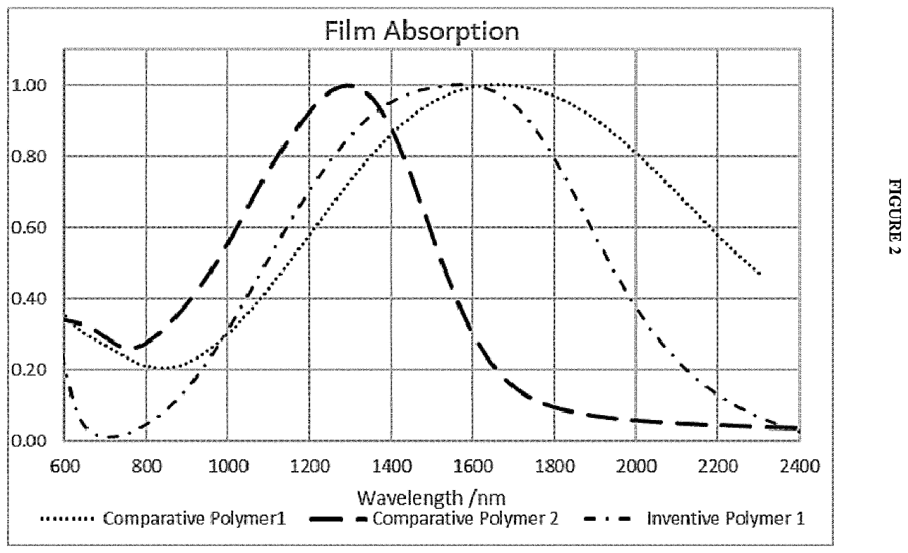

FIG. 2 shows absorption of Polymer Example 1 and Comparative Polymers 1 and 2, illustrated below. The x:y molar ratio for all polymers is 1:1.

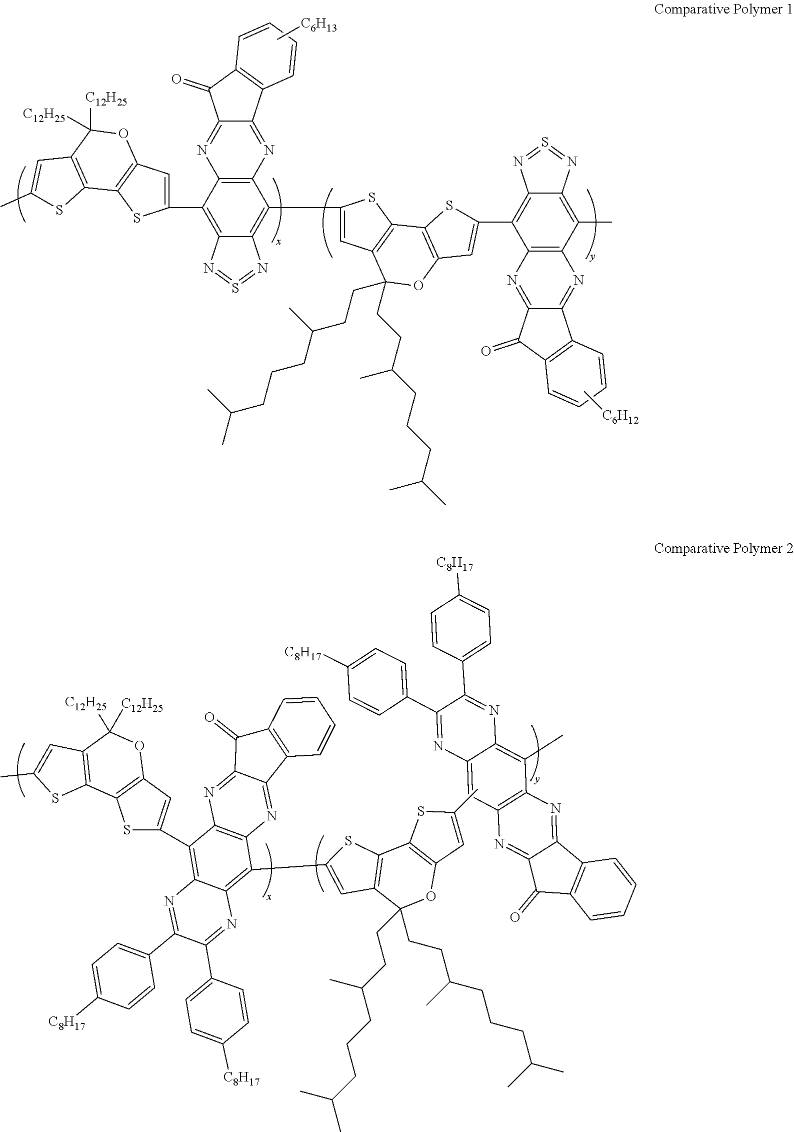

### HOMO-LUMO Data

HOMO and LUMO values of Polymer Example 1 and Comparative Polymers 1 and 2 are set out in Table 1.

TABLE 1

| Polymer | HOMO (eV) | LUMO (eV) |
|---|---|---|
| Polymer Example 1 | −4.81 | −3.55 |
| Comparative polymer 1 | −4.91 | −3.79 |
| Comparative polymer 2 | −4.65 | −3.73 |

### Modelling Examples

All modelling as described in these examples was performed using Gaussian09 software available from Gaussian using Gaussian09 with B3LYP (functional).

It will be understood that modelling data is not measured in the same way as SWV as described herein.

HOMO and LUMO levels for acceptor (ACC) of model compounds of General Formula 1 were modelled:

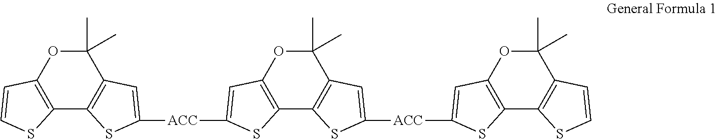

Results are set out in Table 2, in which the illustrated structure shows a donor unit and acceptor group ACC of General Formula (I). S1f corresponds to oscillator strength of the transition from Si (predicting absorption intensity), Eopt is the modelled optical gap.

TABLE 2

| | | LUMO/ | Eg/ | | Eopt/ |
|---|---|---|---|---|---|
| Structure | HOMO/eV | eV | eV | S1f | nm |
| 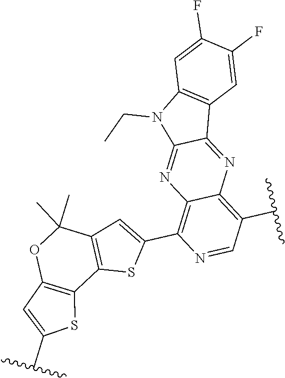 [SMILES: *c1cc2c(s1)-c1sc(-c3ncc(*)c4nc5c6cc(F)c(F)cc6n(CC)c5nc34)cc1C(C)(C)O2] | −4.53 | −2.74 | 1.78 | 1.03 | 829 |
| 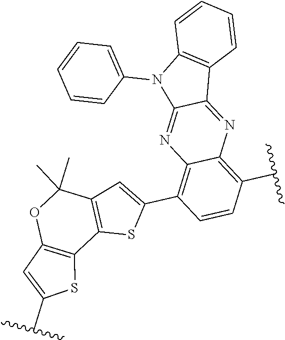 [SMILES: *c1cc2c(s1)-c1sc(-c3ccc(*)c4nc5c6ccccc6n(-c6ccccc6)c5nc34)cc1C(C)(C)O2] | −4.41 | −2.35 | 2.06 | 1.17 | 601 |
| 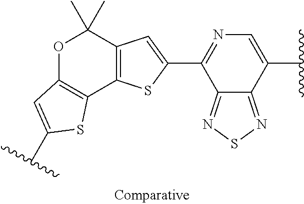 [SMILES: *c1cc2c(s1)-c1sc(-c3ncc(*)c4nsnc34)cc1C(C)(C)O2] | −4.599 | −3.05 | 1.54 | 1.45 | 915 |
| 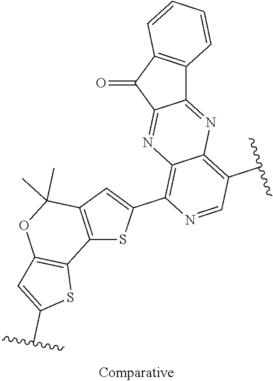 [SMILES: *c1cc2c(s1)-c1sc(-c3ncc(*)c4nc5c(nc34)C(=O)c3ccccc3-5)cc1C(C)(C)O2] | −4.65 | −2.99 | 1.66 | 0.72 | 900 |
| 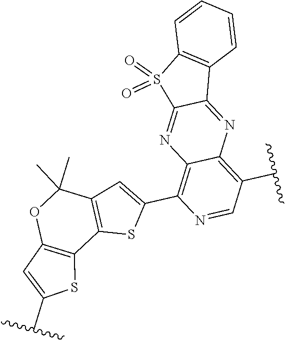 [SMILES: *c1cc2c(s1)-c1sc(-c3ncc(*)c4nc5c(nc34)S(=O)(=O)c3ccccc3-5)cc1C(C)(C)O2] | −4.79 | −3.15 | 1.64 | 0.98 | 912 |
| 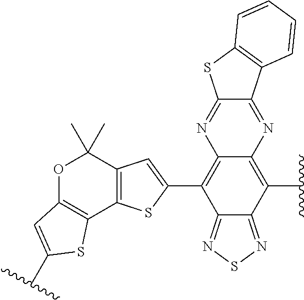 [SMILES: *c1cc2c(s1)-c1sc(-c3c4nsnc4c(*)c4nc5c(nc34)sc3ccccc35)cc1C(C)(C)O2] | −4.31 | −3.36 | 0.95 | 1.29 | 1377 |
| 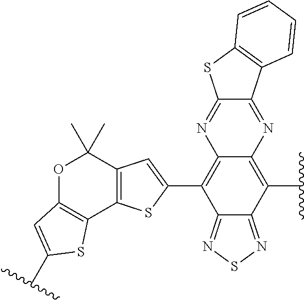 [SMILES: *c1cc2c(s1)-c1sc(-c3c4nsnc4c(*)c4nc5c(nc34)sc3ccccc35)cc1C(C)(C)O2] | −4.31 | −3.36 | 0.95 | 1.30 | 1377 |
| 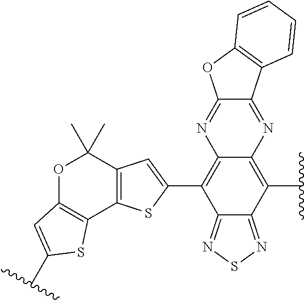 [SMILES: *c1cc2c(s1)-c1sc(-c3c4nsnc4c(*)c4nc5c(nc34)oc3ccccc35)cc1C(C)(C)O2] | −4.27 | −3.33 | 0.94 | 1.31 | 1382 |
| 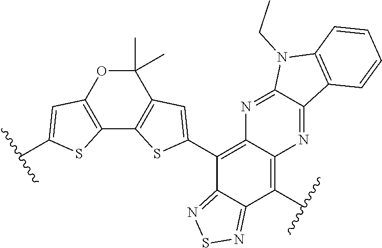 [SMILES: *c1cc2c(s1)-c1sc(-c3c4nsnc4c(*)c4nc5c6ccccc6n(CC)c5nc34)cc1C(C)(C)O2] | −4.18 | −3.14 | 1.03 | 1.28 | 1305 |
| 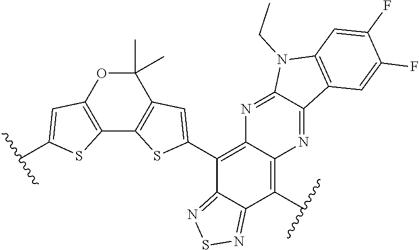 [SMILES: *c1cc2c(s1)-c1sc(-c3c4nsnc4c(*)c4nc5c6cc(F)c(F)cc6n(CC)c5nc34)cc1C(C)(C)O2] | −4.28 | −3.32 | 0.96 | 1.30 | 1376 |
| 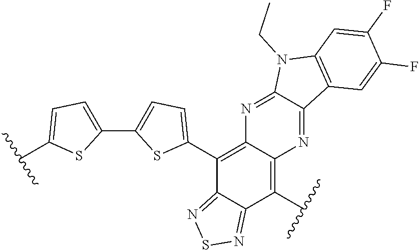 [SMILES: *c1ccc(-c2ccc(-c3c4nsnc4c(*)c4nc5c6cc(F)c(F)cc6n(CC)c5nc34)s2)s1] | −4.51 | −3.28 | 1.22 | 1.02 | 1013 |
| 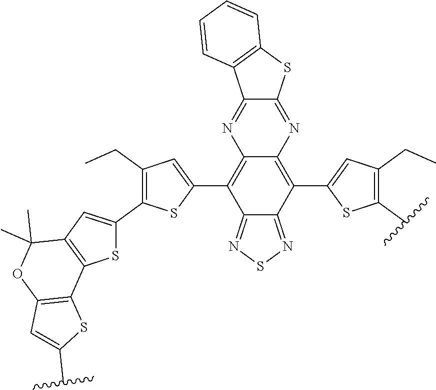 [SMILES: *c1cc2c(s1)-c1sc(-c3sc(-c4c5nsnc5c(-c5cc(CC)c(*)s5)c5nc6sc7ccccc7c6nc45)cc3CC)cc1C(C)(C)O2] | −4.32 | −3.30 | 1.02 | 1.99 | 1430 |
| 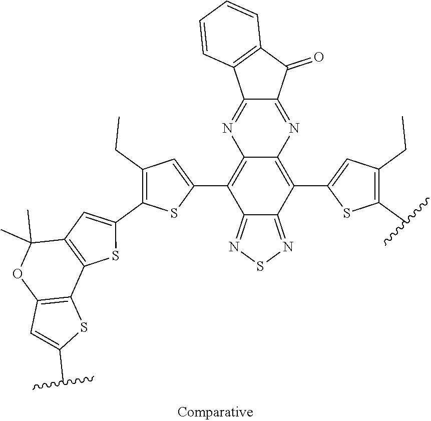 [SMILES: *c1cc2c(s1)-c1sc(-c3sc(-c4c5nsnc5c(-c5cc(CC)c(*)s5)c5nc6c(nc45)-c4ccccc4C6=O)cc3CC)cc1C(C)(C)O2] | −4.39 | −3.38 | 1.01 | 1.66 | 1461 |
| 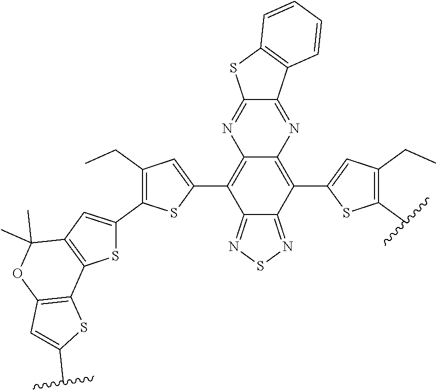 [SMILES: *c1cc2c(s1)-c1sc(-c3sc(-c4c5nsnc5c(-c5cc(CC)c(*)s5)c5nc6c(nc45)sc4ccccc46)cc3CC)cc1C(C)(C)O2] | −4.32 | −3.30 | 1.02 | 1.99 | 1430 |
| 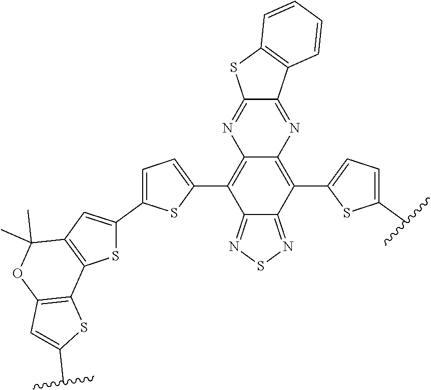 [SMILES: *c1ccc(-c2c3nsnc3c(-c3ccc(-c4cc5c(s4)-c4sc(*)cc4OC5(C)C)s3)c3nc4sc5ccccc5c4nc23)s1] | −4.37 | −3.34 | 1.03 | 2.11 | 1408 |
| 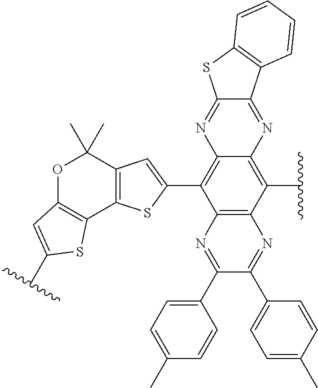 [SMILES: *c1cc2c(s1)-c1sc(-c3c4nc(-c5ccc(C)cc5)c(-c5ccc(C)cc5)nc4c(*)c4nc5c(nc34)sc3ccccc35)cc1C(C)(C)O2] | −4.25 | −2.97 | 1.28 | 1.04 | 1159 |
| 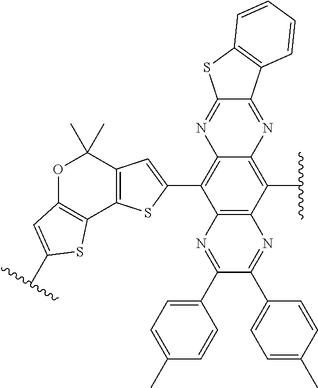 [SMILES: *c1cc2c(s1)-c1sc(-c3c4nc(-c5ccc(C)cc5)c(-c5ccc(C)cc5)nc4c(*)c4nc5c(nc34)sc3ccccc35)cc1C(C)(C)O2] | −4.25 | −2.97 | 1.28 | 1.04 | 1159 |
| 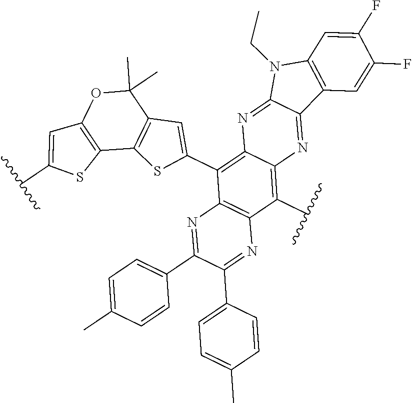 [SMILES: *c1cc2c(s1)-c1sc(-c3c4nc(-c5ccc(C)cc5)c(-c5ccc(C)cc5)nc4c(*)c4nc5c6cc(F)c(F)cc6n(CC)c5nc34)cc1C(C)(C)O2] | −4.27 | −2.88 | 891 | 0.95 | 1078 |
| 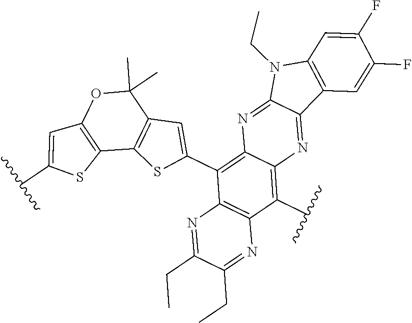 [SMILES: *c1cc2c(s1)-c1sc(-c3c4nc(CC)c(CC)nc4c(*)c4nc5c6cc(F)c(F)cc6n(CC)c5nc34)cc1C(C)(C)O2] | −4.23 | −2.94 | 963 | 1.08 | 1134 |
| 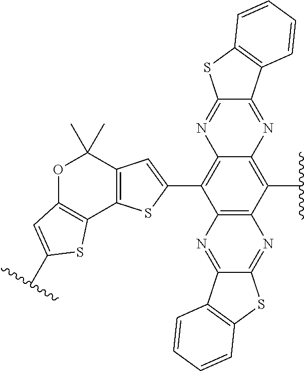 [SMILES: *c1cc2c(s1)-c1sc(-c3c4nc5sc6ccccc6c5nc4c(*)c4nc5sc6ccccc6c5nc34)cc1C(C)(C)O2] | −4.32 | −3.15 | 1.17 | 1.08 | 1244 |
| 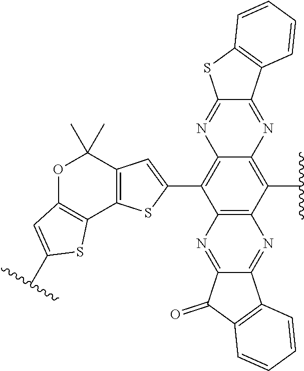 [SMILES: *c1cc2c(s1)-c1sc(-c3c4nc5c(nc4c(*)c4nc6c(nc34)sc3ccccc36)-c3ccccc3C5=O)cc1C(C)(C)O2] | −4.40 | −3.29 | 1.11 | 1.00 | 1312 |
| 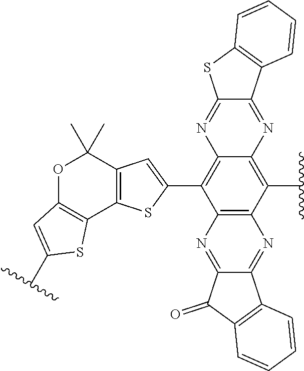 [SMILES: *c1cc2c(s1)-c1sc(-c3c4nc5c(nc4c(*)c4nc6c(nc34)sc3ccccc36)-c3ccccc3C5=O)cc1C(C)(C)O2] | −4.40 | −3.28 | 1.11 | 1.00 | 1312 |
| 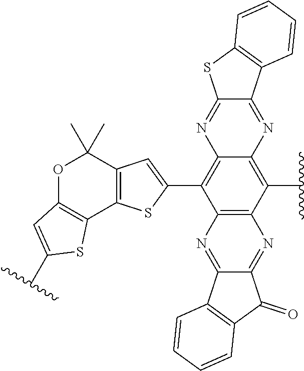 [SMILES: *c1cc2c(s1)-c1sc(-c3c4nc5c(nc4c(*)c4nc6c(nc34)sc3ccccc36)C(=O)c3ccccc3-5)cc1C(C)(C)O2] | −4.38 | −3.30 | 1.15 | 0.73 | 1367 |
| 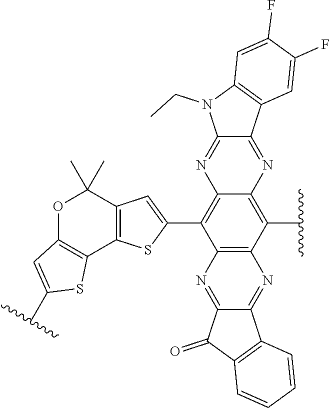 [SMILES: *c1cc2c(s1)-c1sc(-c3c4nc5c(nc4c(*)c4nc6c7cc(F)c(F)cc7n(CC)c6nc34)-c3ccccc3C5=O)cc1C(C)(C)O2] | −4.40 | −3.19 | 1.21 | 0.90 | 1243 |
| 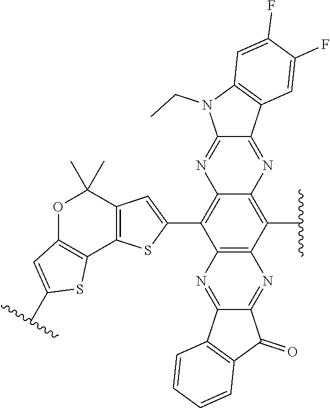 [SMILES: *c1cc2c(s1)-c1sc(-c3c4nc5c(nc4c(*)c4nc6c7cc(F)c(F)cc7n(CC)c6nc34)C(=O)c3ccccc3-5)cc1C(C)(C)O2] | −4.42 | −3.15 | 0.98 | 0.81 | 1183 |
| 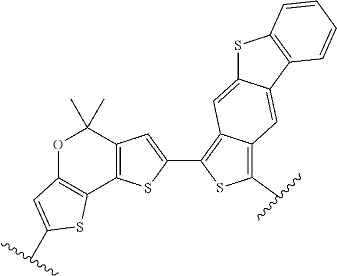 [SMILES: *c1cc2c(s1)-c1sc(-c3sc(*)c4cc5c(cc34)sc3ccccc35)cc1C(C)(C)O2] | −4.25 | −2.92 | 1.33 | 1.09 | 929 |
| 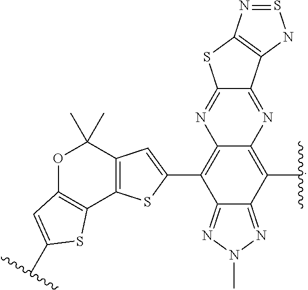 | −4.41 | −3.24 | 1.17 | 1.23 | 1061 |

1. A polymer comprising a donor repeat unit and an acceptor repeat unit wherein the acceptor repeat unit comprise a repeat unit of formula (I): 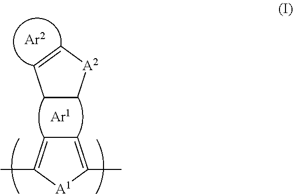
wherein:
A¹ is selected from formula (IIa): O; S; and NR¹ wherein R¹ is H or a substituent: 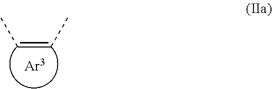
Ar³ is a monocyclic or polycyclic aromatic or heteroaromatic group which is unsubstituted or substituted with one or more substituents;
X¹ and X² are each independently selected from N and CR² wherein R² in each occurrence is H or a substituent with the proviso that at least one of X¹ and X² is selected from N and CR² wherein R² is an electron withdrawing group;
Ar¹ is selected from pyrrole, benzene, pyridine and 1,4-diazine, each of which is unsubstituted or substituted with one or more substituents;
A² is O, S, SO₂, NR¹ or PR¹; and
Ar² is a monocyclic or polycyclic aromatic group which is unsubstituted or substituted with one or more substituents.

2. The polymer according to claim 1 wherein A¹ is a group of formula (IIa) and Ar³ is a monocyclic heteroaromatic group which is unsubstituted or substituted with one or more substituents.

3. The polymer according to claim 2 wherein Ar³ is thiadiazole.

4. The polymer according to claim 2 wherein Ar³ is 1,4-diazine which is unsubstituted or substituted with one or more substituents.

5. The polymer according to claim 1 wherein A¹ is a group of formula (IIa) and Ar³ is a polycyclic heteroaromatic group which is unsubstituted or substituted with one or more substituents.

6. The polymer according to claim 5 wherein Ar³ is a group of formula (III): 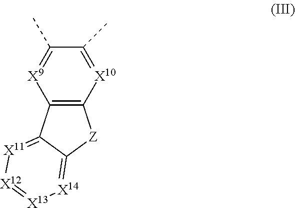
wherein X⁹ and X¹⁰, are each independently selected from N and CR³ wherein R³ in each occurrence is independently H or a substituent;
X¹¹, X¹², X¹³ and X¹⁴ are each independently selected from N and CR³ with the proviso that at least one of X¹¹, X¹², X¹³ and X¹⁴ is CR³; and
Z is selected from O, S, SO₂, NR¹, PR¹, C(R⁴)₂, Si(R⁴)₂ C═O, C═S and C═C(R⁵)₂ wherein R⁴ in each occurrence is independently H or a substituent and R⁵ in each occurrence is an electron-withdrawing group.

7. The polymer according to claim 1 wherein the repeat unit of formula (I) has formula (Ia): 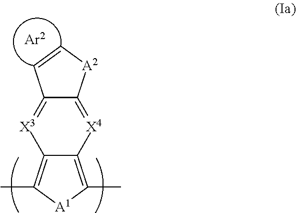
wherein X³ and X⁴ are each independently selected from N and CR³ wherein R³ in each occurrence is independently H or a substituent.

8. The polymer according to claim 7 wherein X³ and X⁴ are each N.

9. The polymer according to claim 1 wherein Ar² is a six-membered aromatic or heteroaromatic group wherein each ring atom is selected from C and N and wherein Ar² is unsubstituted or substituted with one or more substituents.

10. The polymer according to claim 9 wherein the repeat unit of formula (I) has formula (Ib): 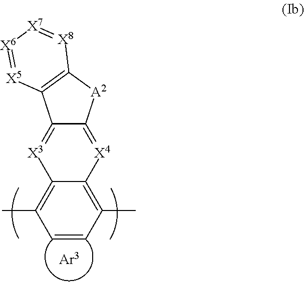
wherein X³ and X⁴ are N; and X⁵, X⁶, X⁷, X⁸, X⁹ and X¹⁰ are each independently selected from N and CR³ wherein R³ in each occurrence is independently H or a substituent.

11. (canceled)

12. The polymer according to claim 1 wherein the donor repeat units are selected from one or more units of formulae (IVa)-(IVt): 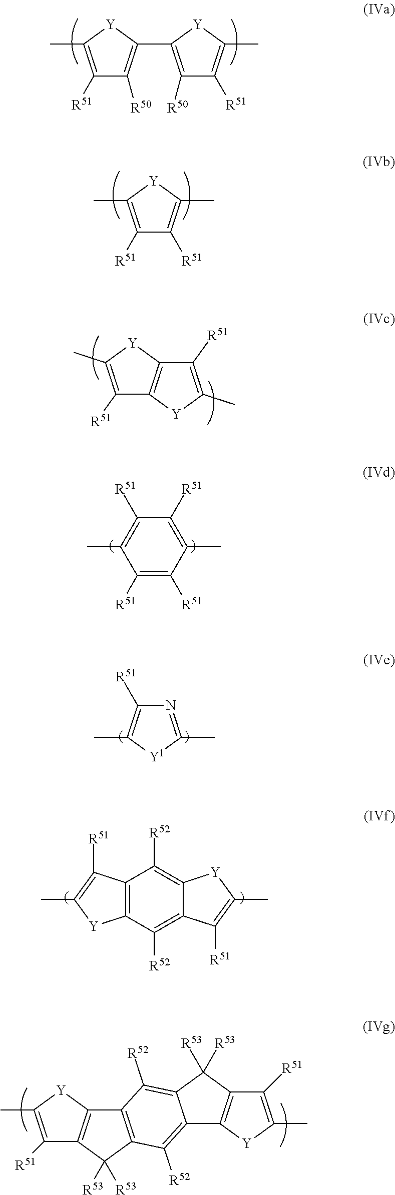 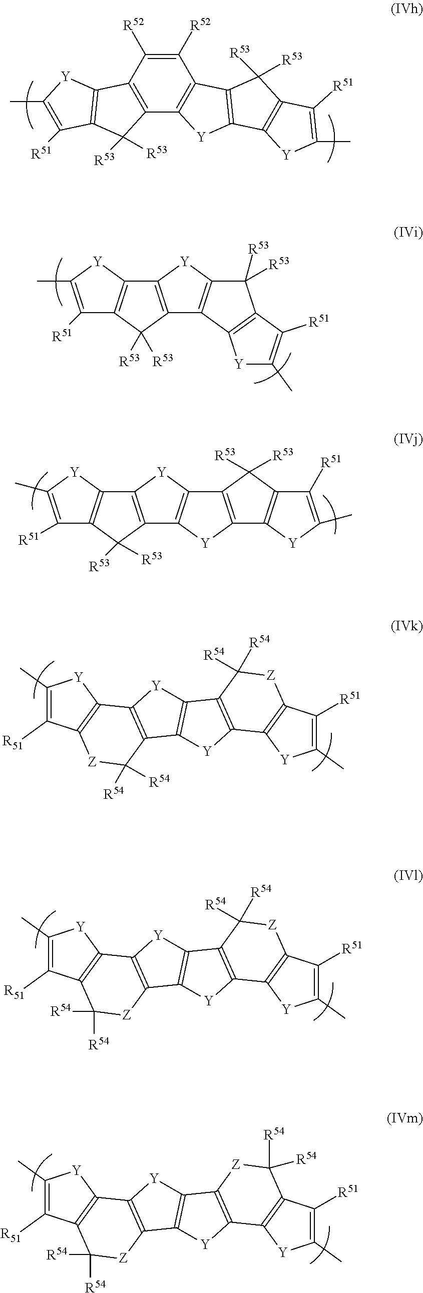 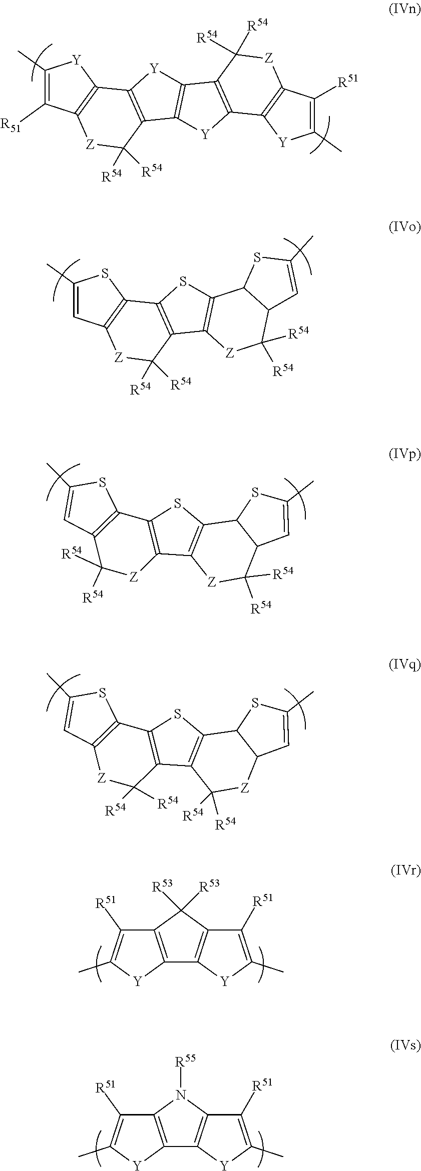 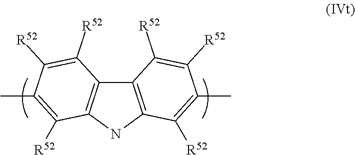
wherein Y and Y¹ in each occurrence is independently O or S, Z¹ in each occurrence is O, S, NR⁵⁵ or C(R⁵⁴)₂; R⁵⁰, R⁵¹, R⁵² R⁵⁴ and R⁵⁵ independently in each occurrence is H or a substituent wherein R⁵⁰ groups may be linked to form a ring; and R⁵³ independently in each occurrence is a substituent.

13. (canceled)

14. The polymer according to claim 12 wherein the donor repeat unit is a repeat unit of formula (IVa) in which the R⁵⁰ groups are linked to form a repeat unit of formula (IVa-1):

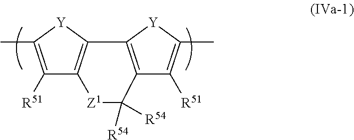

15. The polymer according to claim 1 wherein the polymer has an absorption peak of greater than 1000 nm.

16. A composition comprising an electron donor and an electron acceptor wherein the electron donor is a polymer according to claim 1.

17. An organic electronic device comprising an active layer comprising a polymer according to claim 1.

18. An organic electronic device comprising an active layer comprising a polymer according to claim 1, wherein the organic electronic device is an organic photoresponsive device comprising a bulk heterojunction layer disposed between an anode and a cathode and wherein the bulk heterojunction layer comprises a composition comprising an electron donor and an electron acceptor wherein the electron donor is the polymer according to claim 1.

19. An organic electronic device according to claim 18 wherein the organic photoresponsive device is an organic photodetector.

20. A photosensor comprising a light source and an organic photodetector according to claim 19, wherein the photosensor is configured to detect light emitted from the light source.

21. The photosensor according to claim 20, wherein the light source emits light having a peak wavelength of >1000 nm.

22. (canceled)

23. A formulation comprising a polymer according to claim 1 dissolved or dispersed in one or more solvents.

24. (canceled)
